(19) United States
(12) Patent Application Publication
(10) Pub. No.: US 2006/0229505 A1
(43) Pub. Date: Oct. 12, 2006

(54) Method and system for facilitating respondent identification with experiential scaling anchors to improve self-evaluation of clinical treatment efficacy

(76) Inventors: James C. Mundt, Sauk City, WI (US); David J. Katzelnick, Madison, WI (US); John H. Greist, Madison, WI (US); Heidi K. Moore, Middleton, WI (US)

Correspondence Address: Intellectual Property Department;DEWITT ROSS & STEVENS S.C., Suite 401, 8000 Excelsior Drive, Madison, WI, 53717-1914, US

(21) Appl. No.: 11/400,466

(22) Filed: Apr. 7, 2006

Related U.S. Application Data
(60) Provisional application No. 60/669,516, filed on Apr. 8, 2005.

(51) Int. Cl. A61B 5/00; A61B 13/00

(52) U.S. Cl. 600/300; 600/558; 600/559; 128/920

(57) ABSTRACT

A computer-based interviewing method for assessing mental and/or cognitive illness in a human subject is described. The method includes determining one or more personal characteristics of the human subject to be interviewed. The personal characteristics can include gender, age, nationality, ethnicity, accent, dialect, educational level, religion, etc. The subject is then presented with vocal or visual stimuli to which the subject responds. The vocal or visual stimuli are presented in one or more corresponding personal characteristics of the subject determined earlier (e.g. using a voice and/or an animated image and voice that corresponds to one or more of the personal characteristics). The subject's responses are compiled into a programmable computer and analyzed by a pre-selected test protocol. An alphanumeric value is then generated which corresponds to the presence and/or severity of the mental or cognitive illness in the subject tested.

### CROSS-REFERENCE TO RELATED APPLICATIONS

Priority is claimed to provisional application Ser. No. 60/669,516, filed Apr. 8, 2005, which is incorporated herein.

### BACKGROUND

Treatment outcomes in antidepressant medication trials have traditionally used clinician-administered rating scales such as the Hamilton Depression Rating Scale (HAMD) (Hamilton, 1960), the Montgomery-Asberg Rating Scale (MADRS) (Montgomery & Asberg, 1979), and the Inventory of Depressive Symptomatology (IDS) (Rush et al., 1996). Recently, these measures have received increased scrutiny due to the rising rate of failed clinical trials (Khan & Brown, 2001; Walsh et al., 2002). The reliability and validity of clinician assessment depends largely upon the training and expertise of the raters administering the assessments. Methodological problems such as functional unblinding of raters that may compromise randomization blinds (Greenberg et al., 1992) and inflation of baseline severity measures to meet study enrollment goals (DeBrota et al., 1999; Kobak et al 2000) may contribute to current concerns that factors exogenous to the unbiased assessment of depression severity and treatment response may influence study results (Robinson & Rickels, 2000). It is safe to assume that these same concerns exist when assessing the severity and treatment response of other mental illnesses and/or cognitive disorders.

An alternative to the use of clinician assessments for measuring treatment outcomes is the use of patient self-reported measures of depression severity (Edwards et al., 1984). The use of computer technology to elicit self-report measures has been suggested as a possible means to address current problems in the conduct of randomized clinical trials (Greist et al., 2002). The procedural standardization of computer-based assessments may contribute to more reliable assessments, thus improving subject selection, promoting greater disclosure of personally sensitive information, and controlling clinician biases that may arise due to treatment unblinding or expectancy sets. Computer automated versions of the HAMD have been developed and validated for both desktop (Kobak et al., 1990) and interactive voice response (IVR) applications (Kobak et al., 2000). Paper-based self-report versions of the IDS and the Quick Inventory of Depressive Symptomatology (QIDS) have been developed and validated (Trivedi et al., 2004), as has a version of the MADRS (Svanborg & Ashberg, 2001). In nonpsychotic major depressive disorder (MDD) outpatients without overt cognitive impairment, clinician assessment of depression severity using either the QIDS (clinician-administered version) or the HAMD may be successfully replaced by either the self-report or IVR version of the QIDS (Rush et al., 2006).

There are quite a few United States patents that describe methods or devices for diagnosing the psychological condition of a human subject. For example, U.S. Pat. No. 6,053,866, to McLeod, describes a method of diagnosing a psychiatric disorder in a patient. The method involves two distinct sets of questions, and exposes patients to case studies based upon the patient's answers to the first set of questions. At its heart, the method described in the McLeod '866 patent is a sort of self-executing, self-diagnostic test. In short, if the McLeod '866 method functions as disclosed, there is no need for a psychiatrist to make any diagnosis at all; the method would automatically generate a diagnosis. The test questions utilized in McLeod's approach can be presented in writing, or via a computer interface.

U.S. Pat. No. 6,334,778, to Brown, describes a network-based system for diagnosing mental illnesses or conditions from a distance. Brown's system is a remote monitoring tool. The Brown patent indicates that the system described therein provides for “flexible and dynamic querying of the patients.” (See the '778 patent at col. 4, line 55.) The Brown '778 patent, however, does not disclose matching any characteristic of the interviewing process to any characteristic of the subject being interviewed.

U.S. Pat. No. 6,425,764, to Lamson, describes immersing the subject in a virtual reality environment that includes “scoring procedures for quantitatively analyzing the medical condition of the patient.” (See, for instance, Example 3 of the Lamson '764 patent at Example 3, starting at the top of column 19.)

U.S. Pat. No. 6,607,390, to Glenn et al., describes a method for gathering clinical data in studies relating to mood disorders. The method is a “point-and-click”-type interactive assessment that is repeated over a period of time (thereby generating a longitudinal assessment). The system is a self-assessment prompted by visual input from a computer screen, not a vocal input.

U.S. Pat. No. 6,795,793, to Shayegan et al., describes a method for comparing a large collection of data to a chosen benchmark. The method, for example, can be used to gauge the reliability of a test giver.

U.S. Pat. No. 6,165,126, to Merzenich et al., describes a computer-implemented method for assessing depression in a human subject. The approach described is reiterative in nature. A first computer-implemented assessment is performed, which assessment yields an initial numerical index indicative or reflective of the patient's present mental state. If the initial index is greater than a pre-set level, the assessment is repeated after a pre-defined period of time passes. If the index, however, is less than the pre-set level, the patient is treated using computer-implemented interactive behavioral training.

U.S. Pat. No. 6,322,503, to Sparhawk, Jr. describes a method of diagnosing, tracking, and treating depression. At its core, the method described in the Sparhawk patent is a method to determine whether a human subject is suffering from depression by asking a series of questions regarding depressive symptoms (e.g., sleeplessness), the amount of psychotropic medications being taken, and additional questions. The questions are phrased so as to elicit a numerical answer (from 0 to 10) wherein 0 represents the non-existence of the queried symptom and 10 represents the most severe manifestation of the symptom.

### SUMMARY

A shortcoming of the prior art methods as they apply to diagnosis of psychological conditions is that the methods tend to focus on a binary diagnosis of a given condition. That is, the methods tend to render a binary “present” or “not present” decision with respect to the condition, rather than a graded measure with ordinal and/or interval properties. The Merzenich et al. patent. for example, describes following up with a treatment step once the binary diagnostic step has shown that the patient suffers from depression. None of the earlier patents, however, describe any attempt to customize first-person presentations of information, prompts, or questions to patients, based on the personal characteristics of each individual patient, so as to enhance identification with symptoms described and thereby promote rating accuracy. After all, a critical first step in diagnosing and treating mental illness is to gauge accurately the mental status of the patient who is to be treated.

Thus the invention is directed to a computer-implemented method for assessing mental or cognitive status in a human subject. In the preferred embodiment, the method comprises determining at least one personal characteristic of the human subject to be assessed. The personal characteristic may be selected from any identifiable personal characteristic that can be conveyed to the subject via sight or sound. In other words, the personal characteristic may itself be an identifiable or perceivable vocal or visual characteristic of the subject, or may be conveyed via a vocalized statement or visual presentation. For example, the term “personal characteristic” includes, without limitation, gender, age, hair color, eye color, weight, nationality, ethnicity, race, religion, accent, dialect, style of dress, hair style, bodily decorations or lack thereof (e.g., jewelry, tattooing, body piercing), and educational level. The subject is then presented with vocal stimuli, visual stimuli, or both vocal and visual stimuli, to which the subject responds. Of critical importance in the present invention is that the vocal and visual stimuli are presented in a voice, or in a voice and an image (a live-action moving image or an animated image), that corresponds to at least one personal characteristic of the subject as determined earlier. The subject's responses are compiled into a programmable computer. The responses may be of any type, without limitation, such as a recorded narrative response; a numerical response; a binary response either agreeing with or disagreeing with the presented stimuli; a ternary response indicating that the subject feels less than (or worse than) the presented stimuli, greater than (or better than) the presented stimuli, or the same as the presented stimuli, etc. The responses provided by the subject are then analyzed by means of the programmable computer to assess, or measure, the mental or cognitive status of the subject.

The output generated by the programmable computer may comprise an alphanumerical value corresponding to the mental or cognitive status of the subject.

In another version of the invention, it is preferred that the personal characteristic to be used is the gender of the subject and wherein only vocal stimuli (and no other type of stimuli) are presented to the subject. The vocal stimuli presented to the subject correspond to the gender of the subject— thus a female subject would hear a vocal stimulus presented in a woman's voice, while a male subject would hear a vocal stimulus presented in a man's voice.

The vocal stimuli and/or visual stimuli may be presented to the subject by any means now known or developed in the future for conveying audio and/or audiovisual information. For example, and without limitation, the vocal and/or visual stimuli may be presented in person (by a clinician of the appropriate personal characteristics), telephonically (land-line phone, cell phone, satellite phone, etc, including video telephony), or via computer (with the stimuli being stored locally or transmitted to the computer via a local-area network (LAN), a wide-area network (WAN), wireless network (WIFI), and/or a global computer network, such as the Internet).

In the preferred embodiment, the stimuli presented to the subject comprise a series of carefully constructed, first-person statements that comprise, engender, or otherwise embody an accepted protocol for assessing mental illness (e.g., depression, obsessive-compulsive disorder, etc.). A host of such protocols exist, as noted in the background section. In the preferred embodiment (non-limiting), protocol items are selected from (but not limited to) the group consisting of the Children's Depression Rating Scale-Revised, Inventory of Depressive Symptomatology, the Hamilton Depression Rating Scale, and the Montgomery-Asberg Rating Scale. In these scales, the stimuli are “anchoring descriptions” to which the subject responds. The ultimate output is a numerical identifier that corresponds to the mental state of the subject. In another embodiment of the invention, compiled responses are recordings of the subject's vocal responses to structured prompts. These recordings are then used as the customized stimuli to which the subjects later respond. In other words, in the subject's own voice and personal selection of words is recorded in response to a structured series of audio or audio/visual stimuli with which the subject is subsequently asked to identify. Here, the ultimate output of the process is the subjects' responses to a compiled series of recordings of the subject's own thoughts, in the subject's own voice, which are played back to the subject during or after a clinical trial, thereby to aiding in the evaluation of the treatment efficacy of treatments being tested.

In other versions of the invention, the stimuli are matched with a series of personal characteristics of each respondent, such gender, age, and ethnicity of the subject. The vocal and visual stimuli are then presented to the patient, with the vocal and visual stimuli corresponding to the gender, age, and ethnicity of the subject. The stimuli may also comprise responses compiled from the subject to prompts provided to the subject, wherein the responses comprise audio or audiovisual recordings of the subject's own voice or voice and image. These recordings are then presented to the subject as the customized stimuli (to prompt further responses from the subject).

### DETAILED DESCRIPTION OF THE INVENTION

Abbreviations and Definitions:

The following abbreviations and defined terms are used herein. Terms not ascribed a definition herein take their accepted definitions in the field of psychological, psychiatric, and/or medical diagnosis of humans.

CDRS-R=Children's Depression Rating Scale-Revised.

CGI-S=Clinical Global Impression scale for severity (CGI-S). “Computer” or “programmable computer” means any programmable device for manipulating data, now known or developed in the future. The term “computer” explicitly includes, without limitation, microprocessor devices, hand-held devices (e.g., programmable cell phones, personal digital assistants [PDA's], hand-held cellular Internet devices, and the like), notebook and laptop computers, personal computers, workstations, mainframe computers, supercomputers, and the like (acting alone, acting in concert with one another, and acting in concert with other devices such as hardware (e.g., ROM chips), software, and storage devices (e.g. RAM, hard disks, etc.)).

E-SAD=Exemplar Standardized Assessment of Depression.

HAMD=Hamilton Depression Rating Scale.

IDS=Inventory of Depressive Symptomatology.

IVR=Interactive Voice Response.

MADRS=Montgomery-Asberg Depression Rating Scale.

MDD=Major Depressive Disorder.

MERET®=Memory Enhanced Retrospective Evaluation of Treatment (MERET® is a registered trademark of Healthcare Technology Systems, Inc., Madison, Wis.).

PGI-I=Patient Global Impression of Improvement Scale.

PGI-S=Patient Global Impression of Severity Scale.

QIDS=Quick Inventory of Depressive Symptomatology.

RCT=Randomized Clinical Trial.

SD=Standard Deviation.

A starting point for the present invention was to determine whether equivalence could be confirmed in a controlled study as between clinician-based assessment of conventional tests (such as the HAMD, the MADRS, and the CDRS-R, and self-reported measures of the QIDS) versus computer-automated self-reported versions of these scales. In the process of this determination, it was discovered that customization of the computer-administered stimuli, incorporating personal characteristics of the subjects, enhanced personal identification of the subjects with the stimuli and thereby promoted better clinical assessments of the mental and cognitive states of the subjects. Example 1, below, was performed to investigate the reliability and validity of an IVR version of the MADRS, as compared to concurrent clinician assessments using the same MADRS format. Example 2 addresses a similar study for the E-SAD, while Example 3 addresses another assessment protocol, Memory Enhanced Retrospective Evaluation of Treatment. Regardless of the test protocol utilized, the present invention prompts a response from each patient using a stimulus (a voice or a voice plus a real-life or animated motion picture) whose audio or audio/visual characteristics are customized to personal characteristics of the respondent. By personalizing stimuli used to elicit specific responses from each patient, the responses provided by each patient more accurately reflect (and therefore are more truly indicative of) the patient's mental condition at the time the protocol is run.

It is much preferred that the invention be implemented in an IVR format, or a multimedia format incorporating visual graphics (especially for children or adolescents). While not being bound to any particular mechanism or phenomenon, it is believed that the IVR or multimedia format (wherein the subject responds to a series of prompts offered by recorded voice and/or graphical images) can limit the variability and unknown factors that could unduly influence clinician-administered versions of tests such as HAMD, MADRS and others. Thus, the IVR voice that presents prompts to which patients respond can be made to match, for example, the gender and approximate age of the subject being assessed. For example, a Hispanic, English-speaking subject might be presented with vocal stimuli presented in English with a Hispanic accent. Similarly, the vocal stimuli might be accented to reflect even more precise geographic origins of the subject—for example, the vocal stimuli could be inflected with a specific regional accent exhibited by the subject (e.g., the distinctive coastal tidewater accent of Virginia, or the patois of the Louisiana gulf coast, etc.)

The utility of the current invention is particularly notable in the context of clinical studies of efficacy for psychotropic drugs and even more notable in the context of clinical trials of efficacy for psychotropic drugs wherein the test subjects are children and/or adolescents. The high number of failed pediatric antidepressant clinical trials clearly highlights the need for greatly improved tools to measure efficacy in younger patients. See Emslie et al., 2005. Mental illnesses of all sorts are particularly difficult to measure quantitatively. Unlike, say, cancer or diabetes, diseases whose initial state and whose response to any given treatment can be measured with exquisite sensitivity, mental illnesses are not so easily amenable to objective measurements of severity and remission. A refractory cancer is a simple condition to measure quantitatively: the tumor does or does not grow larger after treatment. The same certainty does not apply, however, to any number of equally crippling mental disease states, such as depression, obsessive-compulsive disorder, etc.

One version of the invention is thus directed to a computer-based method for assessing mental and/or cognitive illness in a human subject. The method comprises first determining one or more personal characteristics of the human subject to be evaluated. These personal characteristics are preferably selected from the group consisting of one or more of gender, age, nationality, ethnicity, accent, dialect, educational level, and religion. The subject is then presented with a series of vocal and/or vocal and visual stimuli that require some type of response by the subject. The vocal or visual stimuli are presented in a voice and/or an animated image and voice that incorporate one or more of the personal characteristics of the subject determined a priori to facilitate personal identification before a response is given.

The responses provided by the subject are then compiled into a programmable computer for subsequent analysis. The responses provided by the subject can be analyzed using any type of consistent scale, or parameter set, or protocol now known or developed in the future (e.g., HAMD, MADRS, QIDS, etc.) In the preferred version of the invention, an alphanumerical value that corresponds to the presence and/or severity of the mental or cognitive illness in the subject tested is thus generated.

An advantage of the system is that it generates, in a highly predictive and reproducible fashion, a value the correlates quite closely with the actual mental state of the subject interviewed. When the method is administered over time, it also provides an extremely valuable “diary” of the subject's progress (or lack of progress). This “diary” of self-reported data is highly valuable both to the subject and to the clinician. See Example 3.

The voice-response method of the present invention can be administered by any means now known or developed in the future. Thus, the method can be administered telephonically, via the Internet or other global communication network, via a broadcast medium, etc. Any type of programmable computer can compile the response. As noted above, the term “programmable computer,” designates any type of device capable of storing and manipulating data, either via analog or digital technology, and includes, without limitation, microprocessors, personal computers, mainframe computer, and the like.

In one version of the invention, designated Exemplar Standardized Assessment of Depression (“E-SAD”), the method presents live-action or animated clips of subjects (including children) expressing intrapersonal feeling states in first-person language. For sake of ease in standardizing the assessment protocols, animated clips are preferred because the facial expressions can be very tightly controlled. E-SAD uses multimedia, animated stimuli designed to enhance personal identification with the subjects, and computer processing of the responses to facilitate efficient scaling of the symptom severity measures. The animated exemplars possess multiracial and multiethnic characteristics (e.g., dark hair and eyes), emotive facial expressions, and gender-specific characteristics, such as hair length and style, to match the respondent's gender. In the preferred version, the voice of the lip-synched exemplar character corresponds to the gender of the respondent, the age of the respondent, and the ethnicity of the respondent to promote personal identification. Hair and skin color may correspond as well. In short, any number of personal characteristics, based on the demographics of each individual subject in the study group, can be employed to promote the individual's personal identification with the animated exemplar characters.

In this version of the invention, after watching and listening to a set of exemplar expressions of a specific symptom of depression at discrete levels of severity, the subject compares his own internal feeling state to select the exemplar that best matches his internal feeling state. Video clips of an experienced pediatric clinician may optionally be interspersed with the exemplar character to provide instructions and guidance to the subjects as they progress through the assessment procedure. If included, these clips are preferably programmed to play automatically between the sets of exemplar animations and at appropriate times throughout the assessment to encourage, guide, and instruct the subject. For child subjects (and where possible), it is also preferred that a tandem assessment, using the same exemplar characters, be used to collect symptom severity ratings from parents or other primary caregivers. The same exemplar clips should be used in the tandem assessments. After completing the ratings across all the depression domains, the software generates a report (the report being generated according to known protocols, such as HAMD, MADRS, QIDS, etc., or any pre-defined set of parameters based on the exemplars utilized and the responses elicited from the test subjects). The data are stored electronically.

For example, using first-person facial and verbal expressions, an animated character provides exemplars to serve as rating anchors for each of any number of domains at each of several levels of severity manifestations (depending on the protocol being implemented). For example, when QIDS is applied to assessing children, 17 various symptom items are probed using the anchored descriptors; the adult QIDS panel includes only 16 symptom items. The severity levels for symptom manifestations are modeled on established anchors currently used by the QIDS (so as to provide at least nominal comparability with the conventional QIDS scoring and interpretation).

For a child subject, animations clips preferably show the head and shoulders of a gender-matched youth, and the character's mouth is lip-synched with customized audio files that are also gender-matched to the E-SAD respondents. The character makes natural facial expressions consistent with the expressed feelings. The audio files likewise contain suitable emotive and affective qualities. The audio scripts are written and recorded with concordant expression of emotion to concisely exemplify the domain and severity. Each clip lasts roughly 10 to 15 seconds. A sample audio script for the most severe sad mood anchor might include, “I feel sad all the time. Everyone tells me to cheer up, but I can't; I'm just too sad. I can't take all this.” Within the user interface, a replay button is provided to enable the subjects to view each exemplar as often as needed before subjects are required to respond to the exemplar. Generally, the subjects are instructed to respond whether the examplar is similar or dissimilar to their current physical, mental, or emotional state.

### EXAMPLES

The following Examples are included solely to provide a more complete description of the invention disclosed and claimed herein. The Examples are not intended to limit the scope of the invention in any fashion.

Thus, a starting point for the present invention was to determine whether the clinician-based assessment of the MADRS and self-reported measures of the QIDS could be confirmed to be comparable or equivalent in a controlled study. Example 1, below, was performed to investigate the reliability and validity of an IVR version of the MADRS using the invented techniques described herein, would affirm equivalence with concurrent clinician assessments using the same MADRS protocol.

The preferred embodiment of the invention is directed to a computer-based interviewing method for assessing mental and/or cognitive illness in a human subject. The method comprises first determining one or more personal characteristics of the human subject to be interviewed. These personal characteristics are preferably selected from the group consisting of one or more of gender, age, nationality, ethnicity, accent, dialect, educational level, and religion. The subject is then presented with a series of vocal and/or visual stimuli that require some type of response by the subject. The vocal or visual stimuli are presented in a voice and/or an animated image and voice that correspond to one or more of the personal characteristics determined a priori.

The responses provided by the subject are then compiled into a programmable computer for subsequent analysis. The responses provided by the subject can be analyzed using any type of consistent scale, or parameter set, or protocol now known or developed in the future (e.g., HAMD, MADRS, IDS, etc.) An alphanumerical value that corresponds to the presence and/or severity of the mental or cognitive illness in the subject tested is thus generated.

An advantage of the system is that it generates, in a highly predictive and reproducible fashion, a value the correlates quite closely with the actual mental state of the subject interviewed. When the method is administered over time, it also provides an extremely valuable “diary” of the subject's progress (or lack of progress). This “diary” of self-reported data is highly valuable both to the subject and to the clinician.

The voice-response method of the present invention can be administered by any means now known or developed in the future. Thus, the method can be administered telephonically, via the internet or other global communication network, via a broadcast medium, etc. The response can be compiled by any type of programmable computer. As used herein, the term “programmable computer,” designates any type of device capable of storing and manipulating alpha-numeric data, either via analog or digital technology, and includes, without limitation, microprocessors, personal computers, mainframe computer, and the like.

### EXAMPLE 1

### Use of Dynamically Adaptive Vocal Stimuli to Obtain Reliable and Valid Self-Reported Montgomery-Asberg Depression Rating Scale Data

Sixty subjects (26 men and 34 women) aged 22 to 64 years (Mean=42.7 years; SD=10.6 years) were recruited through newspaper advertisements by the Department of Psychiatry at the University Health Network, Toronto, Canada. The sample was 80% Caucasian, and 74% had at least some college. Subjects who endorsed symptoms of depression during a brief telephone screen were invited to participate. They subsequently signed informed consent documents and were enrolled in the study. Study methods and materials were reviewed and approved by the University Health Network Research Ethics Board (Toronto, ON).

Subjects completed both the clinician-administered, face-to-face MADRS and the IVR self-report version of the MADRS in a counter-balanced order at the research office. For the IVR MADRS, patients began by providing an overall rating of their self-perceived severity for each of the ten MADRS depression items (listed in Table 1) from 0 (no symptom present) to 6 (extremely severe). After providing this rating, the patients were presented with an appropriate anchoring description in a voice matched to the gender of the patient. That is, women heard a female voice and men heard a male voice. The anchoring description (“anchor” ) was spoken with an affective intonation corresponding to the severity of the symptom being assessed. The patients were then asked whether his or her internal feeling state was “less severe,” “equally severe,” or “more severe” than the presented anchor. The subjects were allowed to listen to the gender-matched anchor as many times as they wished. Patients indicating lesser (or greater) severity than the presented anchor were dynamically provided the next lower (or higher) anchor and allowed to indicate the accuracy of that anchor for describing his or her feelings. Thus, regardless of the initial starting place, each subject was allowed to dynamically titrate up or down the severity scale until the subject felt the anchoring description accurately reflected his or her own feelings, or until the subject indicated a feeling state located between two anchors. If the initial anchoring description accurately reflected the subject's feelings, that anchor point was used to assign a numeric value to the subject's present feelings for that item.

The IVR MADRS uses anchoring descriptions for scale severities of 0, 2, 4, and 6 (the same as the original scale). Patients indicating a greater severity than a first anchor, and a lesser severity than the next higher anchor were assigned scale values of 1, 3, or 5. For example, severity scores for the symptom of “Reported Sadness” (Item 2) were anchored by “I haven't felt sad at all this past week, except when it was appropriate” (score=0); “I feel a bit sad or low but I brighten up without difficulty” (score =2); “I am thoroughly sad or gloomy, but things can make me feel a little bit better at times” (score =4); “I am extremely sad and miserable all the time and cannot snap out of it at all” (score=6).

After completing the clinician-administered MADRS interview and the IVR MADRS interview, an IVR diagnostic interview (Mental Health Screener®-brand) was administered (Kobak et al 1997). Clinicians also completed the Clinical Global Impression Scale for severity (CGI-S), and patients completed the Patient version of the same scale (PGI-S) (Guy 1976). Subjects were paid $50 for their participation. A sub-sample of 20 subjects was reassessed 24 hours later by a different clinician, and repeated the IVR MADRS to evaluate test-retest reliability. These subjects received an additional $50 to compensate for their time.

Fifty of the 60 subjects were diagnosed with a mood disorder by the Mental Health Screener® diagnostic interview (42 with a major depressive episode (MDE), 4 with dysthymia, and 4 with MDE in partial remission). Four subjects were diagnosed with one or more anxiety disorders and two indicated probable alcohol abuse or dependence. Four subjects received no diagnosis from the diagnostic interview.

The mean (±SD) MADRS total scores at the initial assessment were 24.50 (±9.09) for clinician assessment and 25.30 (=9.32) for the IVR assessment. The mean difference of 0.80 (±5.60) did not approach statistical significance, t(59)=1.11, p=0.273, indicating equivalence between the measures. The correlation between clinician and IVR MADRS scores was 0.815, p<0.001. To test for an order effect, separate analyses comparing subjects who received the clinician-administered assessment compared to the IVR assessment first were carried out. This produced equivalent results, indicating that the order of administration was not a factor for either assessment method.

Agreement between methods on individual items and total scores were compared by matched t-tests of mean score differences and intra-class correlation coefficients.

Cronbach's Alpha was computed to assess the internal consistency of the items within both scales. Results of these comparisons are presented in Table 1.

TABLE 1
Comparison of item scores (Mean ± SD) and internal scale reliability
for clinician- and IVR-administered versions of the Montgomery-Asberg Depression
Rating Scale. Sixty subjects were assessed by both methods on the same day in
counter-balanced order

| | | | | Intraclass |
|---|---|---|---|---|
| | | | Difference | Correlation |
| Item | Clinician | IVR | t-test (59 df) | (agreement) |
| Apparent Sadness | 2.30 (1.18) | 2.23 (1.49) | −.348 p = .729 | .398 p = .001 |
| Reported Sadness | 2.77 (1.21) | 3.12 (1.53) | 1.936 p = .058 | .469 p < .001 |
| Inner Tension | 2.58 (1.33) | 2.70 (1.27) | .708 p = .482 | .520 p < .001 |
| Reduced Sleep | 2.85 (1.89) | 2.75 (1.74) | −.629 p = .532 | .769 p < .001 |
| Reduced Appetite | 1.68 (1.62) | 1.90 (1.66) | 1.635 p = .107 | .800 p < .001 |
| Concentration Difficulties | 2.92 (1.36) | 2.90 (1.59) | −.089 p = .930 | .522 p < .001 |
| Lassitude | 2.83 (1.71) | 2.70 (1.58) | −.782 p = .437 | .679 p < .001 |
| Inability to Feel | 2.80 (1.65) | 2.77 (1.59) | −.166 p = .868 | .545 p < .001 |
| Pessimistic Thoughts | 2.63 (1.28) | 3.12 (1.64) | 2.659 p = .010 | .505 p < .001 |
| Suicidal Thoughts | 1.13 (1.42) | 1.12 (1.61) | −.123 p = .903 | .764 p < .001 |
| Total Score | 24.50 (9.09) | 25.30 (9.32) | 1.107 p = .273 | .815 p < .001 |
| Cronbach's Alpha | 0.816 | 0.796 | | |
| (internal consistency) | | | | |

The mean MADRS total scores on the second assessment, 24 hours following the 15 initial assessment, were 24.95 (±7.05) for clinician assessment and 25.30 (±6.50) for the IVR assessment. The mean difference of 0.35 (±5.32) did not approach statistical significance, t(19)±0.29, p±0.772, and the two measures were correlated .694, p±0.001. The test-retest correlations over the two days were 0.904 for the clinician assessments (p<0.001) and 0.850 for the IVR assessments (p<0.001). The mean 20 clinician MADRS score dropped 2.15 (±3.54) points between day 1 and day 2, t(19)=2.71, p=0.014, and was paralleled by a mean IVR MADRS drop of 3.30 (±3.83) across the days, t(19)=3.86, p=0.001. The mean difference in change scores of 1.15 (±4.03) between assessment methods was not statistically significant, t(19)=1.28, p=0.217. The correlation of change scores between methods was .404, which approaches significance (p=0.077), but is not statistically different than 0 in a two-tailed test.

The clinician-administered MADRS scores and the IVR MADRS scores of depression severity converged well with Clinician and Patient Global Impressions at visit 1. The clinician MADRS scores correlated 0.882 and 0.613 with the CGI-S and PGI-S, respectively, while the IVR MADRS scores correlated 0.748 and 0.782 respectively with these same measures. The correlation between CGI-S and PGI-S was 0.652 at visit 1, all p's<0.001. Among the 20 subjects returning for the second visit, the clinician MADRS scores correlated 0.885 and 0.690 with the CGI-S and PGI-S, respectively (p's<0.001), and the IVR MADRS scores correlated 0.474 (p=0.035) and 0.671 (p=0.001) respectively with these same measures. The CGI-S and PGI-S were correlated 0.516 at visit 2, p=0.02.

The data obtained in this Example provide support for the equivalence between the clinician and IVR versions of the MADRS using the inventive methods disclosed herein. The total MADRS scores obtained by each method were statistically equivalent and highly correlated. Scale reliability measures, both Cronbach's Alpha and the 24-h test-retest correlations, were comparable. Scores obtained for nine of the ten individual items were statistically equivalent, although the subjects' self-reported sadness tended toward higher ratings than clinician assessments (p=0.058). Subjects did self-report more severe pessimistic thoughts than reflected in the clinician ratings (p=0.010). The difference may be statistical artifacts (inflated Type 1 error due to the multiple pair-wise comparisons) or reflect real differences between the way clinicians and patients perceive the severity of these symptoms. These minor differences, even if statistically reliable, would not presently indicate a need to revise the IVR MADRS. First, the magnitudes of the item score differences (less than half a point) are unlikely to be clinically meaningful. Second, given the nature of the depression symptoms in question (self-reported sadness and pessimistic thoughts), it is far from clear whether the “gold standard” metric for accurately assessing the true symptom score should be based on the clinicians' or patients' ratings. The total MADRS scores obtained by each method were statistically equivalent and highly correlated. Scale reliability measures, both Cronbach's Alpha and the 24-hour test-retest correlations, were comparable.

The IVR MADRS implementation included several innovative elements, which the present inventors strongly believe contributed to the notable correspondence between methods. Assessment instructions and definitions of the individual items were presented to the subjects in a very structured clinical manner by a highly experienced psychiatrist (co-inventor John H. Greist). First, the voice used to present the phrases that anchored the subjects' self-reported ratings were presented in a different voice—a voice matched to the gender of the subject and spoken with an affective inflection consonant with the emotional content of the anchoring expression. This process was designed to aid the subjects' ability to identify with the descriptive anchors and more accurately determine whether their recent emotional experiences are effectively expressed. Second, the subjects were given an opportunity to indicate whether the emotional intensity of the phrases used to anchor the ratings over- or under-stated their feelings. If so indicated, the anchor phase for the next lower (or higher) rating for that item was presented dynamically and the subjects were given another opportunity to reflect upon the adequacy of that descriptor in describing their emotional experiences. This process permitted subjects to fine-tune, in an adaptive fashion, the self-ratings for each of the MADRS items to match their internal state in a manner quite similar to the method of adjustment used in psychophysical research.

The results ofthis Example are significant because they indicate that customized and personalized delivery of clinical stimuli yields IVR results that closely match those obtained via a clinician-based assessment. In other words, matching the voice of the IVR-presented anchors to the gender of the subjects yielded results that were more accurate in reflecting each subject's true emotional state. In short, matching the voice that presents the questions to the subject by (for example) the gender, age, nationality, ethnicity, accent, dialect, educational level, etc., of the respondent yields results that are more accurate and reflective of each subject's true emotional state. Thus, the present Example shows that customizing the IVR process to use the individual characteristics of each respondent (such as gender, age, nationality, ethnicity, voice, dialect, etc.) improves the accuracy of subjectivejudgments regarding clinical states.

### EXAMPLE 2

### Exemplar Standardized Assessment of Depression

The assessment of depression severity in children and adolescents in clinical trials has also received increased scrutiny. The Children's Depression Rating Scale (CDRS-R) is the currently accepted instrument for evaluating efficacy in clinical trials, relying on clinicians' subjective judgments based on interviews with the child, parent, or other person to obtain ratings of symptom severity relative to anchored descriptors.

Computer-based interviewing techniques for obtaining self-reported depression severity measures directly from adults have been researched for more than 15 years. In 2004, the U.S. Food & Drug Administration announced that interactive voice response (IVR) versions of the HAMD, IDS and QIDS were acceptable primary outcome measures for adult outpatient major depressive disorder clinical trials. The validated techniques from Example 1 (which was specifically directed to adapting the MADRS assessment to a computer-based self-report form using personally customized rating anchors and dynamically adaptive presentation) are applicable to CDRS-R and can be used for the assessment of depression severity among children and/or adolescents using a self-reported auditory and visual format.

The CDRS-R uses anchored descriptors written in the third person to define the severity of symptoms for clinicians to use for rating interviewees' responses. In the present invention, first-person statements that might be made by a typical child or adolescent at a given severity are presented to the child or adolescent for comparison with their current psychological state. For example, a numerical rating of “3” for the CDRS-R social withdrawal item corresponds to the statements: “Does not actively seek out friendships but waits instead for others to initiate a relationship.” And “Occasionally rejects opportunities to play, without having a describable alternative.” In the E-SAD implementation these statements would be expressed in the first person by a child/adolescent as follows (the text being exemplary and non-limiting): “I don't usually try to make friends, but if other kids come up and want to be friends with me it's okay. Sometimes I just don't want to join in with them, even though I really don't have anything else I want to do.” In the present invention, a series of such first-person statements are created for multiple symptoms across the range of severity.

Using multimedia techniques, these first-person expressions are then presented in a manner that maximizes respondent identification. Currently available animation software can create characters with features the same as or similar to characteristics in each respondent (e.g., gender, age, skin tone, ethnicity, eye or hair color, jewelry or lack thereof, religious paraphernalia or lack thereof, etc.) to present age- and gender-matched characters expressing the first-person perspective. (Suitable software for three-dimensional facial animation is commercially available from several sources, including Famous3D (San Francisco, Calif.), Face2Face Animation (Summit, N.J.), and Visage Technologies (Linkoping, Sweden).) The emotive content of the speech files and facial expressions of the animated characters is made to correspond with the affective content of each first-person statement, while simultaneously preserving essential standardization parameters (e.g., wording of the questions or statements, speaking rate, pronunciation, voice timbre, etc.) across the customized character features. Using standardized, but individually tailored, exemplars to present first-person expressions similar to those of a child or adolescent at a particular state of symptom severity, respondents will more accurately render self-ratings of psychological states due to the greater personal identification with the customized expression.

Specifically, after presentation of a first-person statement reflecting a specific severity level on a particular symptom, feature similarity between the animated character and respondent should make reporting that they feel “the same,” “less,” or “more” intensity easier and more accurate. Dynamic, adaptive presentation of other levels of severity can be presented, as described in Example 1.

The multimedia program (which can be downloaded or administered over the Internet or other computer network or installed from a storage medium [e.g., a compact disk, hard-drive, etc.]) operates according to the following four steps:

Demographic parameters, such as the respondent's age, gender, ethnicity, religion (if any), etc. are entered into the program. This information is used to select and/or customize the appropriate character and speech files for the assessment. In the preferred embodiment, other information, such as alphanumeric identification indicia, date, time, location where the test is administered, etc. is also stored in a header file to assist data management.

In the preferred embodiment, a clinical, in silico “narrator” (preferably an adult character and voice) describes the process of listening to statements/expressions with which any given respondent may or may not identify. The “narrator” also provides instructions for responding. The “narrator” also appears between assessments of each symptom domain to describe the relevant construct for the next set ofjudgments (such as social withdrawal or low self-esteem). This narrative is provided at different (and age-appropriate) abstraction levels for different age ranges of subjects being assessed. Before proceeding to the symptom ratings, respondents are asked to confirm whether or not they understand the construct. Additional narrative instructions are provided if necessary.

In the preferred embodiment, the respondent is then presented with an animated clip of a child/adolescent (matched on relevant features) making first-person statements that define, or anchor, a specific severity level for that symptom. The respondent is able to replay the expression as many times as needed. The clip can be of any length, but is likely most effective as a concise 10- to 15-second statement that best exemplifies the symptom and severity. An alternative embodiment could use video clips of child actors with characteristics in common with the child/adolescent being assessed to anchor the rating scale.

The respondent then makes a judgment whether his or her own feelings are less severe, equally severe, or more severe than the presented exemplar. Judgments of lesser or greater severity are then followed up with further expressions anchoring other levels of symptom severity. This dynamic, adaptive process for eliciting self-reported ratings of symptom severity using stimuli customized to reflect respondent characteristics is implemented for each domain of depression deemed critical to overall severity.

For example, each symptom domain could be scored on a numerical scale (0 to 4,1 to 10, etc.) For purpose of illustration only, a 7-point scale will be discussed. To achieve 7-point scaling, it is preferred that at least three definitive first-person statement/expressions be formulated to anchor the scale values of 2, 4, and 6. If, for example, the respondent is first presented with the severity anchor that defines a value of 4, the respondent either endorses the expression as matching his own feelings (receiving a rating of 4 and moving on to the next symptom domain) or the respondent replies that his experience is more or less intense than the exemplar presented. If the respondent rated his severity as less intense, the respondent would be presented with an exemplar expression that defines/anchors a severity of 2. Self-ratings of even less severity than the level 2 exemplar receive a symptom severity rating of 1. Personal identification with the level 2 exemplar receives a score of 2, and indications of greater severity receive a rating of 3. Symptom severity ratings of 5, 6, or 7 are obtained by judgments relative to the exemplar expression that anchored a symptom severity rating of 6. The resulting data can be stored locally or centrally, or transmitted to a database or some other remote location over the Internet using secure data transfer protocols. A report summarizing the results and notifying the test administrator of any critical information, such as elevated suicidal ideation, can be generated immediately.

### EXAMPLE 3

### Memory Enhanced Retrospective Evaluation of Treatment

In the same fashion as in Example 1 (MADRS) and Example 2 (E-SAD), the present invention can also be implemented with an assessment method known commercially as Memory Enhanced Retrospective Evaluation of Treatment (MERET®-brand assessments, a registered trademark of Healthcare Technology Systems, LLC).

Many study design elements influence the methodological effectiveness for discriminating the efficacy of treatments in randomized clinical trials (RCTs). Two of the most critical design issues are: (1) selection of the outcome measures to be used for assessing treatment effects; and (2) the source of clinical outcomes data.

Clinical change associated with treatments can be assessed using serial measurement of disease severity to evaluate pre-post treatment differences, or retrospective assessments of perceived change after treatment. Randomized clinical trials typically use serial severity assessment measures—for example, the HAMD, MADRS, and IDS—in antidepressant clinical trials (as mentioned earlier). Retrospective ratings of clinical change, however, such as ratings of global impressions of improvement since the start of treatment are also frequently obtained. A study comparing both approaches for measuring treatment-related change found that retrospective assessments may be more sensitive than serial measures and better reflect patients' satisfaction with the treatments provided (Fischer et al., 1999)

A second factor to consider in assessing treatment efficacy is the source of outcome data. RCTs typically rely upon clinical ratings of the severity of patients' symptoms by trained research staff. The increasing rate of failed antidepressant trials has raised concerns about current RCT assessment methods (Greist et al., 2002; Khan et al., 2002). An alternative to clinical rater data is direct patient-reported outcomes (PROs). The reliability and validity of several patient-reported assessment instruments have been well established and accepted by the Food and Drug Administration as outcome measures for evaluating treatment efficacy for Major Depressive Disorder in outpatient trials. The debate regarding methodological equivalence or superiority between clinician-rated severity scales or PROs remains an unresolved research issue.

A fundamental problem with asking patients to make retrospective judgments about clinical improvement after treatment is the need for them to recall accurately experiences before treatment. The reconstructive nature of personal memory makes unaided, accurate recall of past experiences increasingly difficult with the passage of time. Patients' retrospective judgments of change relative to experiences that occurred weeks or months in the past are undoubtedly influenced by how well they remember the past experiences. Memory aids that facilitate remembrance of past experiences using personally relevant recognition cues facilitate retrospective judgments of change, relative to judgment methods that rely solely on direct experiential recall.

In 2002, a pilot study was conducted to explore a concept entitled Memory Enhanced Retrospective Evaluation of Treatment (Mundt et al., 2003). This assessment protocol is marketed under the MERET trademark by Healthcare Technology Systems, LLC (Madison, Wis.). The 2002 study assessed the feasibility of using interactive voice response (IVR) telephone technology to allow patients to record personal descriptions of their baseline emotional and physical experiences, and the affect of those feelings on their daily functioning, in an antidepressant RCT. Several weeks later the personalized baseline recordings were played back to the patients, before asking them to rate perceived clinical change on a 7-point Patient Global Impression of Improvement (PGI-I) (Guy, 1976) scale: 1=Very Much Better; 2=Much Better; 3=A Little Better; 4=Unchanged; 5=A Little Worse; 6=Much Worse; 7=Very Much Worse. The patients also rated how helpful hearing the baseline recordings was for making retrospective ratings of clinical change.

The pilot study results demonstrated that MERET procedures were feasible and practical as a technique for providing patients with personalized experiential anchors to facilitate subsequent ratings of relative clinical change. As expected, patients' ratings of the helpfulness of hearing the MERET recordings was correlated with how much they actually recorded about their baseline experiences.

The MERET-brand assessment, however, is not a simple voice diary. The subjects are prompted to respond to specific, structured questions and prompts directly relevant to the physical, mental, and functional impairments associated with clinical manifestations of psychopathology. Measures of the elicited response, such as how long they speak, are used to prompt additional speech to optimize the subsequent utility of the procedure. The personalized recordings that are obtained represent the ultimate customization of stimuli designed to enhance personal identification with the expressed psychological state. The recording elicitation procedures result in stimuli that match the subject's traits regarding gender, age, nationality, ethnicity, accent, dialect, educational level, and religion exactly. Subsequent use of these stimuli to obtain self-reported ratings of clinical change since that time maximizes the capability of the patient to identify with the expressed psychological state, specific to the clinical symptoms important for assessing mental health and psychological well-being.

By way of example, a clinical study to evaluate a psychotropic drug, using the present invention as a means to evaluate the test subjects, might proceed as follows:

The study requires a series of office visits, roughly about 6 to 10. Between study entry at Visit 1 and baseline acquisition at Visit 2 (one to four weeks), patients do not receive study drug and they discontinue any medications they might have been taking prior to study entry. Beginning at Visit 2, patients are randomized to receive placebo or an investigational compound and are then evaluated weekly at the investigators' site offices for the stated length of the study. Patients discontinue taking the study drug on the penultimate visit, and then there is an ultimate follow-up visit. During the weeks when the study drug is being administered, 50% of the patients are randomized to receive placebo, 25% of patients are randomized to receive an initial period of placebo, followed by the test drug administered at a first dosage, and 25% of patients are randomized to receive an initial period of placebo, followed by the test drug administered at a second dosage (which is either higher or lower than the first dosage).

During each office visit patients call an IVR system to provide self-report data. During baseline call at Visit 2, patients are prompted to record personal descriptions to one or more structured prompts (an exemplary list is presented below). This procedure results in the creation of individually customized, personally identifiable stimuli containing individual characteristics of each patient. Patients are instructed that the purpose of the recordings is to serve as a personal memory aid to recall their current experiences more accurately during and after treatment, and encouraged to express their current physical, mental, and functional state as completely as possible Exemplary prompts for soliciting the MERET records in the study are as follows: (These prompts may be presented in a format “customized” to each particular patient and/or medical condition being treated.) “Please describe your physical condition during the past week. Think about whether you've been feeling ill or tired, or had pain anywhere in your body. Describe your physical condition as completely as you can.” (This prompt probes the patient's physical condition.) “Please describe your mental condition during the past week. Think about the thoughts, feelings, and emotions you've had. Describe your mental condition as completely as you can.” (This prompt probes the patient's mental condition.) “Please describe how your physical condition and/or mental condition have affected your general ability to function during the past week. Think about your ability to work, manage your home, get along with others, and participate in leisure activities. Describe your functioning as completely as you can.” (This prompt probes the patient's functional condition.)

Following each prompt, patients are allowed to speak for as long as they wish, or up to a pre-set maximum amount of time (e.g., 3 minutes, 5 minutes, 10 minutes, etc.). If the total duration of the recorded speech following a prompt is too terse, say less than 20 seconds, patients are encouraged to describe their experiences in greater detail. Again, the encouraging prompt may provided in a voice matched to the characteristics of the patient, as noted earlier. In the preferred embodiment, the patients are given an opportunity to playback each recording and add any additional comments they may care to voice. While the patients are given an opportunity to review each recording, in the preferred embodiment they are not allowed to delete or re-record their initial descriptions.

At one or more subsequent visits after treatment randomization, patients are presented with the personally customized recordings elicited at baseline. After listening to their individual descriptions of their prior experiences, they are asked to provide a rating of clinical change since that time with respect to being unchanged, better, or worse. If the patient indicates clinical improvement or worsening, he or she is asked to rate the extent of change as “a little” “much” or “very much”. Seven-point Patient Global Impression of Improvement (PGI-I) ratings from 1 (very much improved) to 7 (very much worse), with a ratings of 4 representing “unchanged” are obtained.

The significance of this Example is that if patients cannot tell whether or not they have improved after treatment, any discussion about the effectiveness of the treatment provided must be suspect. After several weeks or months of treatment, patients may not be able to recall how they were feeling before they started treatment. Remembering what they had for lunch on any given day one week ago (not an easy task) may be easier than accurately recalling intrapersonal experiences of several weeks or months ago. Highly salient, emotionally laden experiences are more easily recalled than mundane, typical daily experiences, but these day-to-day experiences are critical indicators of both physical and psychological health. The present invention provides a method for creating individually customized stimuli (personalized recordings) to more accurately recall the day-to-day experiences firmly anchored in a time prior to treatment. By obtaining patients' descriptions in their own words, of their emotional, physical, and functional experiences at the beginning of treatment, the customization of the stimuli to which they subsequently respond by providing ratings maximizes personal identification with the recorded experiences. The personal identification and enhanced recollection facilitates better comparison with current clinical states, and consequently enhances the accuracy of ratings of clinical change. Moreover, because the prompts may be presented in customized format, the responses elicited by the prompts more accurately reflect the true physical, mental, and functional states of the subjects, and (perhaps even more importantly) the change over time in those states over the course of a treatment blinded study.

The present invention thus asks patients to describe in their own words, their emotional, physical, and functional experiences at the beginning of treatment, in response to vocal prompts presented in a voice (or voice and appearance for audiovisual prompts) customized to the characteristics of each patient. The very process of verbalizing their feelings may facilitate deeper intrapersonal processing of their current clinical status. The descriptions provided are recorded for playback after treatment, preferably before asking the patients to make retrospective judgments about clinical change. Using patients' intrapersonal descriptions of their own experiences to anchor pretreatment clinical states allows them to express the symptoms of greatest distress and personal salience to them. Subsequently hearing their own descriptions, in their own words and voices, represents the ultimate stimulus customization allowing the patients direct access to their thought processes and internal experiences that existed at the time the recordings were made. The selection of words, the tone of voice, the affect and the points of hesitation have considerable value for personal insight from which more accurate judgments of current clinical states can be rendered. Just as each individual is uniquely qualified to read his or her own handwriting, each person is likely best able to understand the meaning and content of their own speech—both spoken and unspoken.

### REFERENCES

DeBrota D, Demitrack M, Landin R, Kobak KA, Greist JH, Potter W. A comparison between interactive voice response system-administered HAM-D and clinician-administered HAM-D in patients with major depressive episode, National Institute of Mental Health, New Clinical Drug Evaluation Unit, 39th Annual Meeting. Boca Raton, Fla., 1999.
Edwards BC, Lambert MJ, Moran PW, McCully T, Smith KC, Ellingson AG. A meta-analytic comparison of the Beck Depression Inventory and the Hamilton Rating Scale for Depression as measures of treatment outcome. British Journal of Clinical Psychology 1984;23 :93-99.
Emslie, G. J., Heiligenstein, J. H., Wagner, L. S., et al. (2002). Fluoxetine for acute treatment of depression in children and adolescents: A placebo-controlled, randomized clinical trial. Journal of the American Academy of Child and Adolescent Psychiatry, 41, 1205-1215.
Fischer D, Stewart AL, Bloch DA, et al. Capturing the patient's view of change as a clinical outcome measure. JAMA 1999; 282:1157-62.
Greenberg RP, Greenberg MD, Bornstein RF, Fisher S. A meta-analysis of antidepressant outcome under “blinder” conditions. Journal of Consulting and Clinical Psychology 1992;60:664-669.
Greist JH, Mundt JC, Kobak K. Factors contributing to failed trials of new agents: can technology prevent some problems? Journal of Clinical Psychiatry 2002;63(Suppl 2):8-13.
Guy W. ECDEU Assessment Manual for Psychopharmacology, revised. Rockville, Md.: National Institute of Mental Health, US Dept. of Health, Education, and Welfare publication ADM 1976;76:338.
Hamilton M. A rating scale for depression. Journal of Neurology, Neurosurgery and Psychiatry 1960;23:56-62.
Khan A, Brown WA The placebo enigma in antidepressant clinical trials. Journal of Clinical Psychopharmacology 2001;21:123-125.
Khan A, Leventhal RM, Khan SR, Brown WA. Severity of depression and response to antidepressants and placebo: an analysis of the Food and Drug Administration database. J Clin Psychopharmacol 2002; 22(1):40-5.
Kobak KA, Mundt JC, Greist JH, Katzelnick DJ, Jefferson JW. Computer assessment of depression: automating the Hamilton Depression Rating Scale. Drug Information Journal 2000;34:145-156.
Kobak KA, Reynolds WM, Rosenfeld R, Greist JH. Development and validation of a computer-administered version of the Hamilton Depression Rating Scale. Psychological Assessment 1990;2:56-63.
Kobak KA, Taylor LH, Dottl SL, et al. A computer-administered telephone interview to identify mental disorders. Journal of American Medical Association 1997;278:905-10.
Montgomery SA, Asberg M. A new depression scale designed to be sensitive to change.

British Journal of Psychiatry 1979; 132:382-389.
Mundt JC, Brannan S, DeBrota DJ, et al. Memory Enhanced Retrospective Evaluation of Treatment: MERET. Proceedings of the National Institute of Mental Health, New Clinical Drug Evaluation Unit, Boca Raton, FL: National Institute of Mental Health, June, 2003:183.
Robinson DS, Rickels K. Concerns about clinical drug trials. Journal of Clinical Psychopharmacology 2000;20 :593-596.
Rush AJ.: Development and Evaluation of the Quick Inventory of Depressive Symptomatology (QIDS) (Clinician-Rated, Self-Report, and Interactive Voice Response Assessment), 44th Annual Meeting New Clinical Drug Evaluation Unit (NCDEU). Phoenix, Ariz., 2004.
Rush AJ, Bernstein IH, Trivedi MH, Carmody TJ, Wisniewski S, Mundt JC, Shores-Wilson K, Biggs MM, Nierenberg AA, & Fava M. (2006). An Evaluation of the Quick Inventory of Depressive Symptomatology and the Hamilton Rating Scale for Depression: A Sequenced Treatment Alternatives to Relieve Depression Trial Report. Biological Psychiatry. Mar 15;59(6):493-501.]
Rush AJ, Gullion CM, Basco MR, Jarrett RB, Trivedi M. The inventory of depressive symptomatogy (IDS): psychometric properties. Psychological Medicine 1996;26, 477-486:477-486.
Svanborg P, Ashberg M. A comparison between the Beck Depression Inventory (BDI) and the self-rating version of the Montgomery Asberg Depression Rating Scale (MADRS). Journal of Affective Disorders 2001;64:203-216.
Trivedi MH, Rush AJ, Ibrahim HM, et al. The Inventory of Depressive Symptomatology, Clinician Rating (IDS-C) and Self-Report (IDS-SR), and the Quick Inventory of Depressive Symptomatology, Clinician Rating (QIDS-C) and Self-Report (QIDS-SR) in public sector patients with mood disorders: a psychometric evaluation. Psychological Medicine 2004;34:73-82.
Walsh BT, Seidman SN, Sysko R, Gould M. Placebo response in studies of major depression. Journal of American Medical Association 2002;287:1840-1847.

What is claimed is:

1. A computer-implemented method for assessing mental or cognitive status in a human subject, the method comprising:
(a) determining at least one personal characteristic of the human subject to be assessed;
(b) presenting to the subject vocal stimuli, visual stimuli, or both vocal and visual stimuli to which the subject responds, wherein the vocal and visual stimuli are presented in a voice, or in a voice and an image, that correspond to the at least one personal characteristic determined in step (a);
(c) compiling responses provided by the subject into a programmable computer; and
(d) analyzing by means of the programmable computer the responses provided by the subject to assess the mental or cognitive status of the subject.

2. The method of claim 1, wherein step (a) comprises determining at least one personal characteristic selected from the group consisting of gender, age, hair color, eye color, weight, nationality, ethnicity, race, accent, dialect, educational level, and religion.

3. The method of claim 1, further comprising:
(e) generating an alphanumerical value corresponding to the mental or cognitive status of the subject.

4. The method of claim 3, wherein the alphanumeric value is generated by allowing the subject to respond to one or more descriptive anchors, each anchor having assigned to it a numerical value proportional to severity of a symptom, wherein the subject identifies the descriptive anchor or anchors that most closely correspond to the subject's condition with respect to the symptom.

5. The method of claim 1, wherein step (a) comprises determining gender of the subject; and step (b) consists of presenting vocal stimuli only to the subject, wherein the vocal stimuli corresponds to the gender of the subject.

6. The method of claim 5, wherein in step (b), the vocal stimuli are presented telephonically or via computer.

7. The method of claim 5, wherein in step (b), the vocal stimuli comprise first-person statements comprising a protocol selected from the group consisting of the Children's Depression Rating Scale-Revised (“CDRS-R”), the Hamilton Depression Rating Scale (“HAMD”), the Montgomery-Asbe'rg Rating Scale (“MADRS”), the Inventory of Depressive Symptomatology (“IDS”), and the Quick Inventory of Depressive Symptomatology (“QIDS”).

8. The method of claim 5, wherein step (a) comprises compiling responses from the subject to prompts provided to the subject, wherein the responses comprise recordings of the subject's own voice; and step (b) comprises presenting to the subject the recordings, wherein the vocal stimuli of step (b) comprise the recordings.

9. The method of claim 5, further comprising:
(e) generating an alphanumerical value that corresponds to the mental or cognitive status of the subject.

10. The method of claim 9, wherein the alphanumeric value is generated by allowing the subject to respond to one or more descriptive anchors, each anchor having assigned to it a numerical value proportional to severity of a symptom, wherein the subject identifies the descriptive anchor or anchors that most closely correspond to the subject's condition with respect to the symptom.

11. The method of claim 1, wherein step (a) comprises determining gender of the subject; and step (b) comprises presenting vocal and visual stimuli to the subject, wherein the vocal and visual stimuli correspond to the gender of the subject.

12. The method of claim 1, wherein step (a) comprises determining gender, age, and ethnicity of the subject; and step (b) comprises presenting vocal and visual stimuli to the patient, wherein the vocal and visual stimuli correspond to the gender, age, and ethnicity of the subject.

13. The method of claim 12, wherein in step (b), the vocal and visual stimuli are presented telephonically or via computer.

14. The method of claim 13, further comprising:
(e) generating an alphanumerical value that corresponds to the mental or cognitive status of the subject.

15. The method of claim 14, wherein the alphanumeric value is generated by allowing the subject to respond to one or more descriptive anchors, each anchor having assigned to it a numerical value proportional to severity of a symptom, wherein the subject identifies the descriptive anchor or anchors that most closely correspond to the subject's condition with respect to the symptom.

16. A computer-implemented method for assessing mental or cognitive status in a human subject, the method comprising:
(a) determining gender of the human subject to be assessed;
(b) presenting to the subject vocal stimuli, visual stimuli, or both vocal and visual stimuli to which the subject responds, wherein the vocal and visual stimuli are presented in a voice, or in a voice and an image, that correspond to the gender of the subject;
(c) compiling responses provided by the subject into a programmable computer; and
(d) analyzing by means of the programmable computer the responses provided by the subject to assess the mental or cognitive status of the subject.

17. The method of claim 16, further comprising:
(e) generating an alphanumerical value corresponding to the mental or cognitive status of the subject.

18. The method of claim 17, wherein the alphanumeric value is generated by allowing the subject to respond to one or more descriptive anchors, each anchor having assigned to it a numerical value proportional to severity of a symptom, wherein the subject identifies the descriptive anchor or anchors that most closely correspond to the subject's condition with respect to the symptom.

19. The method of claim 16, wherein step (b) consists of presenting vocal stimuli only to the subject, wherein the vocal stimuli corresponds to the gender of the subject.

20. The method of claim 19, wherein in step (b), the vocal stimuli comprise first-person statements comprising a protocol selected from the group consisting of the Children's Depression Rating Scale-Revised (“CDRS-R”), the Hamilton Depression Rating Scale (“HAMD”), the Montgomery-Asberg Rating Scale (“MADRS”), the Inventory of Depressive Symptomatology (“IDS”), and the Quick Inventory of Depressive Symptomatology (“QIDS”).

21. The method of claim 16, wherein step (a) comprises compiling responses from the subject to prompts provided to the subject, wherein the responses comprise audio or audiovisual recordings of the subject's own voice or voice and image; and step (b) comprises presenting to the subject the recordings, wherein the stimuli of step (b) comprise the recordings.

22. A computer-implemented method for assessing mental or cognitive status in a human subject, the method comprising:
(a) determining gender of the human subject to be assessed;
(b) presenting to the subject vocal stimuli to which the subject responds, wherein the vocal stimuli are presented in a voice that correspond to the gender of the subject;
(c) compiling responses provided by the subject into a programmable computer;
(d) analyzing by means of the programmable computer the responses provided by the subject to assess the mental or cognitive status of the subject; and
(e) generating an alphanumerical value corresponding to the mental or cognitive status of the subject.

23. The method of claim 22, wherein in step (b), the vocal stimuli comprise first-person statements comprising a protocol selected from the group consisting of the Children's Depression Rating Scale-Revised (“CDRS-R”), the Hamilton Depression Rating Scale (“HAMD”), the Montgomery-Asberg Rating Scale (“MADRS”), the Inventory of Depressive Symptomatology (“IDS”), and the Quick Inventory of Depressive Symptomatology (“QIDS”).

24. The method of claim 22, wherein step (a) comprises compiling responses from the subject to prompts provided to the subject, wherein the responses comprise audio or audiovisual recordings of the subject's own voice or voice and image; and step (b) comprises presenting to the subject the recordings, wherein the stimuli of step (b) comprise the recordings.

25. The method of claim 22, wherein the alphanumeric value is generated by allowing the subject to respond to one or more descriptive anchors, each anchor having assigned to it a numerical value proportional to severity of a symptom, wherein the subject identifies the descriptive anchor or anchors that most closely correspond to the subject's condition with respect to the symptom.
